(19) United States
(12) Patent Application Publication
(10) Pub. No.: US 2010/0121359 A1
(43) Pub. Date: May 13, 2010

(54) HAEMOSTATIC CLIP, DEVICE TO APPLY A HAEMOSTATIC CLIP AND ITS USE

(76) Inventor: Fabio César Atui, Sao Paulo (BR)

Correspondence Address: HAMRE, SCHUMANN, MUELLER & LARSON, P.C., P.O. BOX 2902, MINNEAPOLIS, MN, 55402-0902, US

(21) Appl. No.: 12/598,507

(22) PCT Filed: May 5, 2008
(86) PCT No.: PCT/BR08/00129

§ 371 Date: Dec. 22, 2009

(30) Foreign Application Priority Data
May 3, 2007 (BR) PI 0701909-2

(51) Int. Cl. A61B 17/122

(52) U.S. Cl. 606/158; 606/157

(57) ABSTRACT

The present invention refers to surgical clips as used to occlude tubular bodies. More specifically, the present invention refers to a haemostatic clip to occlude vessels, such as blood vessels, which constructive arrangement solves a range of inconveniences of haemostatic clips as found in the state of the art. According to an embodiment of the present invention, a first segment (11) and a second segment (12) substantially parallel to each other, being said first segment connected to said second segment by a linking element (13) so to allow its application over a tubular body.

### FIELD OF THE INVENTION

The present invention generally refers to surgical clips as used to occlude tubular bodies.

More specifically, the present invention refers to a haemostatic clip to occlude vessels, such as blood vessels, which constructive arrangement that solves a range of inconveniences of haemostatic clips of the state of the art.

### BACKGROUND OF THE INVENTION

Haemostatic clips are largely employed in various surgical modalities with the purpose to stop bleeding in specific areas by means of the occlusion or closing of the blood vessel. They are usually made of resilient material, such as metal or polymeric materials.

The state of the art includes a series of haemostatic clips, such as disclosed in the documents EP 432 692, U.S. Pat. No. 4,217,902, U.S. Pat. No. 4,449,530, U.S. Pat. No. 4,579,118, U.S. Pat. No. 4,602,632, U.S. Pat. No. 4,638,804, U.S. Pat. No. 5,026,382 and U.S. Pat. No. 5,545,377.

These documents disclose different forms of constructive arrangements applied in haemostatic clips, with different shapes and number of components composing them as well.

In all these solutions of the state of the art, clips work by closing vessels transversely over a central axis of the tubular body. In other words, clips have an open end and an opposed closed end, working as a linking member for both arms composing the open end. As a rule, the clip is located transversely to the vessel and one arm is forced against the other in the open end portion, so to compress the blood vessel. The closed end works as a linking element, so to keep the clip unity.

FIG. 1 shows a haemostatic clip (1) as commonly found in the state of the art. As can be noticed, the clip is applied transversely to a vessel (5) when the open ends (2, 3) are compressed against that same vessel, so to press its walls and stop the flow of blood and consequently the bleeding.

However, there is a range of inconveniences linked to haemostatic clips of the state of the art.

Firstly, it is needed to identify and precisely visualize the blood vessel the flow of which should be stopped. This is particularly difficult in a surgical field, in which frequently the space of vision is extremely reduced. Also, due to the bleeding and blood flow in the surgical area, the surgeon cannot exactly define the place or vessel from which the blood flows.

This inconvenience is particularly serious in case of video laparascopic surgeries, in which the surgeon does not have manual access to the bleeding vessel, which may make vessel identification and clip application using devices become difficult. Clips of the state of the art are therefore inefficient.

In an attempt to stop bleeding, not succeeding to precisely visualize the open vessel, the surgeon applies a series of clips, one aside the other, separated by a short distance to each other, with the purpose to cover the whole area suffering a surgical intervention, in order to attempt the stopping of the bleeding, despite the point where bleeding occurs not being precisely identifiable.

However, besides consuming a large quantity of clips and generating post-surgical discomfort in patients, this practice is also inefficient. This is because, despite a large quantity of clips is applied and the surgeon, when applying such clips, attempts to leave a minimum space between them, there may still be bleedings in the spaces existing between one clip and the other.

Such bleedings may occur because smaller vessels existing between one clip and another have not been occluded and continue to bleed, or because, due to lack of visualization in the surgical field (frequently due to the quantity of existing blood), the clip does not fully occlude a vessel, but just part of it, and the bleeding is not fully stopped.

When this kind of inconvenience is identifiable during the surgery, the surgeon may apply an even larger number of clips, consequently causing too high clip consumption and consequent increase in the cost of surgery due to the higher use of material.

In the specific case of hemorrhoid surgery, bleeding is frequently identified just after the surgery, forcing the patient to return to the surgery bed to apply more clips into the region where the surgery was made, thus causing very high discomfort to the patient, be it for bleeding, be it for requiring a new surgery.

Finally, clips of the state of the art cannot be efficiently applied to substitute the “X” suture.

It is therefore an object of the present invention to provide a haemostatic clip that solves the deficiencies of the clips of the state of the art as mentioned above.

### DESCRIPTION OF THE INVENTION

For that purpose, so to solve the inconveniences as mentioned above, among others, the present invention constitutes a haemostatic clip comprising a first segment and a second segment substantially parallel to each other, being said first segment connected to said second segment by a linking element so to allow its application over a tubular body.

Tubular body should include any part of the animal or human body presenting a substantially tubular shape, such as a blood vessel.

The clip of the present invention may be advantageously applied in any angle over the tubular body in an efficient way, differently from clips of the state of the art, which efficiency to stop bleeding is conditioned to the transversal application to the vase.

The clip of the present invention may also present one or more of the following characteristics:

said first segment and said second segment may comprise substantially identical lengths;

said first segment and said second segment may comprise substantially different lengths;

both segments may be substantially linear;

both segments may be substantially curved or correspond to a segment of a circle;

said linking element comprises an arch connecting the substantial middle portion of the first segment to the substantial middle portion of the second segment;

said linking element comprises two arches located at the ends of each segment, so to connect said first segment to said second segment;

said linking element comprises two arches located at the ends of each segment, so to connect said first segment to said second segment, being said arches sloped in an oblique angle over said segments; and

the transversal section of clip segments may be equal or different, with a tubular, circular, square, triangle or hexagonal shape.

Materials composing the clip may be any material suitable such as metal alloys or polymeric materials. In an particular embodiment, biocompatible or bio-absorbing materials for application to human or animal bodies are used.

### BRIEF DESCRIPTION OF THE DRAWINGS

Enhancements and effects of the present invention will be evident to those skilled in the art from the detailed description as presented below, with reference to the attached drawings, given only as illustrations of specific embodiments of the invention. The drawings are schematic and their dimensions and proportions may not correspond to reality, since they only aim to didactically illustrate the invention, not imposing any limitations than those of the attached claims, wherein:

FIGS. 2A to 2C represent, respectively, a perspective, front and side view of a clip of a first embodiment of the present invention;

FIGS. 3A to 3C represent, respectively, a perspective, front and side view of a clip of a second embodiment of the present invention;

FIGS. 4A to 4C represent, respectively, a perspective, front and side view of a clip of a third embodiment of the present invention; and

FIGS. 5A to 5C represent, respectively, a perspective, front and side view of a clip of a fourth embodiment of the present invention.

### DESCRIPTION OF PARTICULAR EMBODIMENTS

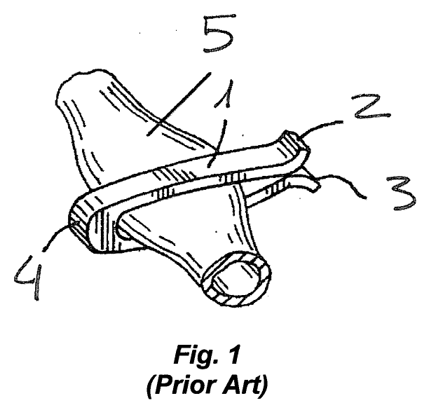

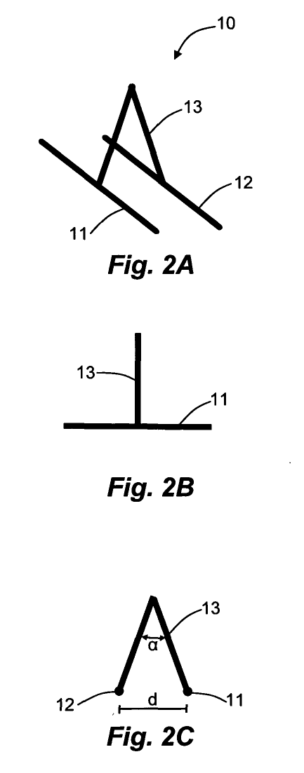

FIGS. 2A to 2C show a haemostatic clip (10) according to a first embodiment of the present invention. The clip comprises a first segment (11) and a second segment (12). The segments are substantially parallel to each other and are connected to each other by a linking element (13). According to the embodiment as shown, the linking element (13) is an arch with substantially equal thickness to that of the segments, connected to the substantial middle portions of each segment.

As shown in the figures, the clip is in its open position, i.e. before being applied to occlude a vessel.

The arch (13) operates as a linking element, and may be folded in a given angle (α). Said angle (α) may vary, but should be comprised between approximately 10° and 180°, particularly between 15° and 90° and more particularly between 20° and 75°. However, the value of the angle is not relevant to the scope of the present invention, as long as there is a distance (d) between the segments of particularly at least 1 mm.

The full length of the arch may also vary according to the size of the clip and its application, and should be comprised between 1 mm and 100 mm, particularly between 2.5 mm and 50 mm and more particularly between 5 mm and 25 mm.

Particularly, both segments (11, 12) have the same size, but may present different lengths, depending on the type of application. The length of each segment may also vary according to the type of application and should be comprised between 1 mm and 25 mm, particularly between 2.5 mm and 15 mm, and more particularly between 5 mm and 15 mm.

Both the segments and the arch may comprise a cross-section of tubular, circle, square, triangle, hexagonal or any other appropriate shape for the mechanic resistance and resilience of the haemostatic clip. The shape of the cross-section of the segments and the arch is not relevant for the scope of the present invention.

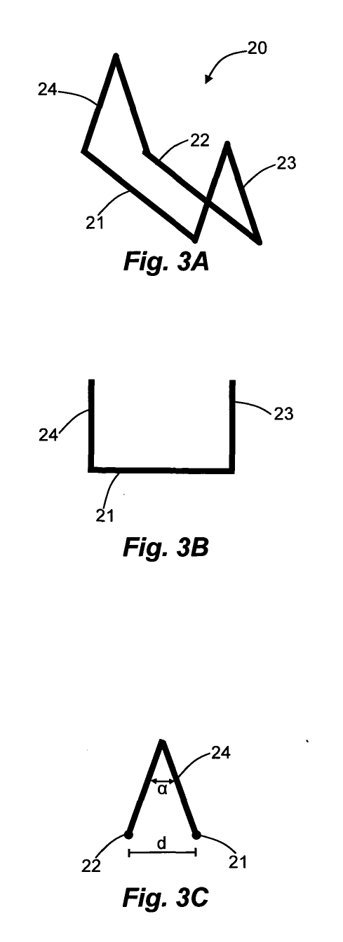

FIGS. 3A to 3C show a second embodiment of the clip of the present invention. In this embodiment, the clip (20) also comprises two substantially parallel segments (21, 22). As a linking element, however, two arches (23, 24) located at the ends of each segment (21, 22) are used.

Just like in the first embodiment, both arches may be folded at their middle portion in an angle (α), when the clip is in its open position. Said angle (α) may also vary, as long as a distance (d) of particularly at least 1 mm is kept between segments.

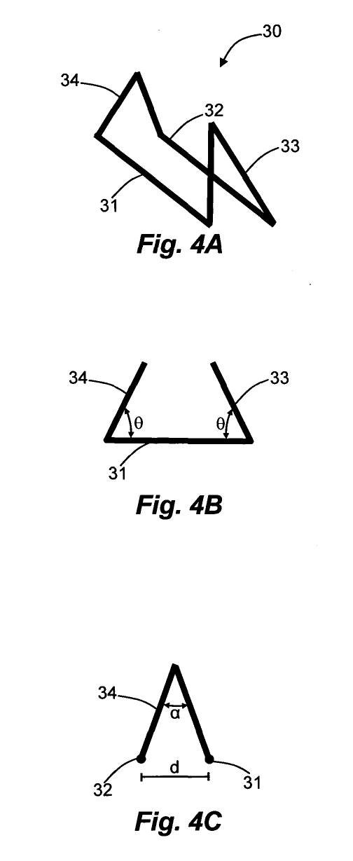

FIGS. 4A to 4C show a third embodiment of the clip (30) of the present invention. This embodiment is very similar to the one as shown in FIGS. 2A to 2C, but the arches (33, 34) operating as a linking element between the segments (31, 32) are sloped in an oblique angle (θ) over said segments, and not substantially orthogonally located, as in the previous embodiments. This embodiment has the advantage of successively applying various clips, one aside the other in a curved way, so that the linking arches (33, 34) do not interfere when a clip is adjacent to the other.

The oblique angle (θ) formed between arches (33, 34) and segments (31, 32) may vary and is preferably comprised between 0° and 90°, more particularly between 15° and 75°, and even more particularly between 20° and 45°.

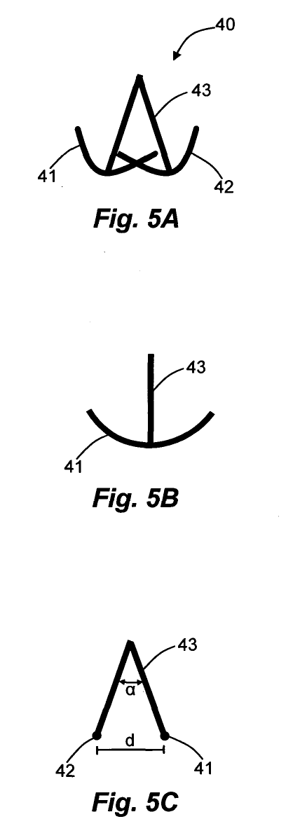

FIGS. 5A to 5C disclose a variation of the shape which may be assumed by segments (41, 42). In this case, despite being substantially similar to each other, segments (41, 42) are not linear and may take a curved form or be similar to a circle segment. Despite the clip of said embodiment being represented analogously to FIGS. 2A to 2C, the curved form of the segments (41, 42) may be expanded to other embodiments as already disclosed.

Various materials may be used to manufacture clips of the embodiments of the present invention. Metallic materials may be used, such as, but not limited to, alloys of stainless steel, titanium, aluminum, magnesium, silver, tantalum etc. or their mixtures. Polymeric materials may also be used, such as, but not limited to polyvinyls, methylcellulose, polyethylenes, polystyrenes, polyesters etc. or their mixtures. Biocompatible or bio-absorbing materials, being them metallic, polymeric or composites, for application to the human or animal body are preferably used.

The clip of the present invention may be applied for both human and veterinary use in any surgery modality, be it a conventional, endoscopic, laparoscopic surgery or even any other surgical technique which may come to exist.

The device to apply the clip of the invention may be any conventional device as found in the state of the art, with required adaptations to apply the clip to a human or animal body.

As evident from the detailed description of particular embodiments, the clip of the present invention solves a range of inconveniences as found in clips of the state of the art, since it allows efficient application in any angle over a body in which occlusion is desired, since it covers a larger area. They are different from clips of the state of the art, which for efficient application, need to be substantially transversal to the vessel.

Therefore, the clip of the invention has a better efficiency, especially in video laparoscopic surgeries, in which the access to the region where bleeding should be stopped is difficult and made by means of devices. The clip of the present invention may also be efficiently used to substitute the “X” suture.

It should be understood that, although the invention has been disclosed with relation to its particular embodiments, those skilled in the art may develop variations of design and operational details and expand the above shown object to other types of applications, but not deviating from the principles of the present invention. The attached claims should therefore be interpreted as covering all equivalents included within the scope of the invention.

1. HAEMOSTATIC CLIP comprising a first segment and a second segment substantially parallel to each other, being said first segment connected to said second segment by a linking element to allow its application over a tubular body.

2. The Clip of claim 1, wherein the clip is applied in any angle in relation to said tubular body.

3. The Clip of claim 1, wherein said first segment and said second segment comprise substantially identical lengths.

4. The Clip of claim 1, wherein said first segment and said second segment comprise substantially different lengths.

5. The Clip of claim 1, wherein the segments are substantially linear.

6. The Clip of claim 1, wherein the segments are substantially curved or corresponds to a circle segment.

7. The Clip of claim 1, wherein said first segment and/or said second segment comprise a length between 1 mm and 25 mm, particularly between 2.5 mm and 15 mm, and more particularly between 5 mm and 15 mm.

8. The Clip of claim 1, wherein said linking element is an arch connecting the substantially middle portion of the first segment to the substantially middle portion (12, 42) of the second segment.

9. The Clip of claim 1, wherein said linking element comprise two arches located at the ends of each segment to connect said first segment to said second segment.

10. The Clip of claim 1, wherein said linking element comprise two arches located at the ends of each segment to connect said first segment to said second segment, wherein arches slope in an oblique angle (θ) over the segments.

11. The Clip of claim 8, wherein said arch is folded at its middle portion in an angle (α) comprised between approximately 10° and 180°, particularly between 15° and 90°, and more particularly between 20° and 75°.

12. The Clip of any claim 8, wherein said arch has full length between 1 mm and 100 mm, particularly between 2.5 mm and 50 mm, and more particularly between 5 mm and 25 mm.

13. The Clip of claim 10, wherein said oblique angle (θ) is comprised between 0° and 90°, particularly between 15° and 75°, more particularly between 20° and 45°.

14. The Clip of claim 1, wherein said tubular body being a blood vessel.

15. The Clip of claim 1, wherein the clip has a cross-section that is a tubular, circle, square, triangle or hexagonal cross-section.

16. The Clip of claim 1, wherein the clip is manufactured from an alloy of: stainless steel, titanium, aluminum, magnesium, silver, tantalum or their mixtures.

17. The Clip of claim 1, wherein the clip is manufactured from a polymeric material.

18. The Clip of claim 1, wherein the clip is manufactured from a biocompatible or bio-absorbing material.

19. DEVICE TO APPLY A HAEMOSTATIC CLIP, by applying a clip as defined in claim 1.

20. USE OF A HAEMOSTATIC CLIP as defined in claim 1, wherein the clip is applied to a human or animal body or as a support instrument for a surgery.

21. (canceled)
